(19) United States
(12) Patent Application Publication
(10) Pub. No.: US 2025/0285300 A1
(43) Pub. Date: Sep. 11, 2025

(54) METHOD AND SYSTEM FOR IMAGE REGISTRATION AND VOLUMETRIC IMAGING

(71) Applicant: Nicholas HINDLEY, Elizabeth Bay (AU)

(72) Inventor: Nicholas HINDLEY, Elizabeth Bay (AU)

(21) Appl. No.: 18/863,661

(22) PCT Filed: May 5, 2023
(86) PCT No.: PCT/AU2023/050380

§ 371 Date: Nov. 7, 2024

(30) Foreign Application Priority Data
May 9, 2022 (AU) 2022901230

(51) Int. Cl. G06T 7/33

(52) U.S. Cl. CPC G06T 7/337; G06T 2207/10076; G06T 2207/10081; G06T 2207/20081

(57) ABSTRACT

The invention provides a system for image registration and volumetric imaging of a patient, the system comprising: a first interface adapted to receive 3D and/or 4D images of the patient; a second interface adapted to receive 2D images during a procedure carried out on the patient; and a processing unit wherein the processing unit is adapted to carry out a method comprising the steps of a method for image registration and volumetric imaging.

### TECHNICAL FIELD

The present invention relates to a method and system for image registration and volumetric imaging; and, more particularly, 3D to 2D image registration (or vice versa) using artificial intelligence.

### BACKGROUND

In many scenarios, intelligent systems are used to make decisions about the motion of 3D objects based on videos or images acquired in 2D. Such systems include autonomous vehicles, for example, wherein heavy, rapidly moving automobiles must adapt to the unpredictable motion of pedestrians. Other examples include warehouse packing, where robotic systems are required to efficiently pack objects of different shapes while avoiding collisions and damage to the objects; and image-guided radiotherapy, where radiation beams are required to adapt to tumours and organs-at-risk that are constantly moving due to the heartbeat and respiration.

Image-based navigation is being investigated as a tool for medical procedures and treatments, with the aim of increasing the access to and reduce the cost of reproducible, safe, and high-precision procedures. Image-based techniques avoid the need for specialized equipment and can seamlessly integrate with contemporary workflows. Furthermore, it is expected that image-based navigation techniques will play a major role in enabling mixed reality environments, as well as autonomous and robot-assisted workflows.

Image-based navigation techniques do not require specialized equipment but rely on traditional intra-operative imaging. A central component of the majority of image-guided navigation solutions is image-based 2D/3D registration, which estimates the spatial relationship between a 3D model of the scene (potentially including anatomy and instrumentation) and corresponding 2D interventional images. For example, registration of pre- and intra-interventional data is one of the key technologies for image-guided radiation therapy, radiosurgery, minimally invasive surgery, endoscopy, and interventional radiology.

In image-guided medical procedures, the registration of pre- and intra-interventional data provides the surgeon with information about the position of instruments relative to the planned trajectory, nearby vulnerable structures, and the target object. Examples of pre-interventional data include three-dimensional (3D) computed tomography (CT) and magnetic resonance (MR) images, while the intra-intervention data are typically two-dimensional (2D) ultrasound (US), projective X-ray (fluoroscopy), CT-fluoroscopy, and optical images, or 3D images like cone-beam CT (CBCT) and US, or 3D digitized points or surfaces.

Image guided radiation therapy uses images of a patient's anatomy to identify the location of a treatment target (either directly or relative to a known structure within the body of a patient). These images are treatment planning images obtained prior to radiation delivery and/or intra-treatment images obtain during treatment delivery, and are taken within a treatment room reference frame. These may be different to a treatment planning image reference frame. Significant challenges arise when attempting to locate a target region (or a structure) within the body of the patient that moves, either just prior to, or during the course of radiation treatment. That is, the target moves from its location within the body when the treatment planning image was acquired. Image registration provides the ability to locate a target region within the body by comparing the image content between two or more images.

Various ways of achieving 2D to 3D registration by utilizing acquired 2D images to register 3D volume images include contour algorithms, point registration algorithms, surface registration algorithms, density comparison algorithms, and pattern intensity registration algorithms. These registrations, however, involve complicated computational tasks and each registration generally take several minutes, or upwards of more than 20 minutes to an hour, to perform each 2D to 3D registration. These registration processes may also result in an inaccurate registration after waiting an extensive period of time.

For example, U.S. Pat. No. 9,165,362 discloses a method of 3D-2D registration for medical imaging, the method which comprises: providing a first input interface for acquiring a three dimensional image: b) providing a second input interface for acquiring a fixed two-dimensional image using an imaging system comprising a source and a detector and having an unknown source-detector geometry; c) initializing image transformation parameters and source-detector geometry parameters; d) generating a reconstructed two-dimensional image from the three-dimensional image using the image transformation parameters and the source-detector geometry parameters; e) determining an image similarity metric between the fixed two-dimensional image and the reconstructed two dimensional image; and f) updating the image transformation parameters and the source-detector geometry parameters using the image similarity metric. This process is computationally heavy and requires significant processing time.

U.S. Pat. No. 10,713,801 also discloses a method, comprising: performing, by a processing device, a first image registration between a reference image of a patient and a motion image of the patient to perform alignment between the reference image and the motion image, wherein the reference image and the motion image include a target position of the patient; performing, by the processing device, a second image registration between the reference image and a motion x-ray image of the patient, via a first digitally reconstructed radiograph (DRR) for the reference image of the patient; and tracking at least a translational change in the target position based on the first registration and the second registration. This process is also computationally heavy and requires significant processing time.

In Balakrishnan et al¹, image registration is treated as a problem in unsupervised learning and solutions are produced by a neural network. However, this method is concerned with 2D-2D and 3D-3D image registration. That is, the method described by Balakrishnan et. al. also cannot be used for efficient 2D-3D image registration and volumetric imaging. ¹ Balakrishnan et al. “A Learning Framework for Deformable Medical Image Registration' (IEEE Transactions on Medical Imaging. 2019 Aug 38; 1558-254X(8): 1788-1800).

In treating diseases such as cancer or cardiac arrhythmia, radiation therapy, or radiotherapy, is a particularly important tool. The most common of cancer-related death involve tumours in the thorax and abdomen. However, thoracic and abdominal tumours are under constant flux due to respiration. As patients breathe, irregular changes are unwittingly induced in their internal anatomy. This continuous motion introduces a significant challenge for the safe and effective delivery of radiotherapy as clinical teams attempt to ablate malignant tissue while minimising collateral damage to the surrounding organs-at-risk. However, existing technologies to address this problem require dedicated systems that are not available in most radiotherapy centres around the world. A recent survey of 200 centres across 41 countries found that 71% wished to extend real-time motion management to additional treatment sites, but were hindered by human and financial resources as well as machine capacity.

However, given the slow rotation speeds of linear accelerators relative to breathing motion, it is not feasible to acquire the sufficient 2D projection data to reconstruct clinically useful 3D volumes. To overcome these challenges, several groups have developed 2D-3D image registration and machine learning techniques. For instance, Li et al². proposed the use of principal component analysis (PCA) to construct a compressed representation of lung deformation vector fields (DVFs), which can then be used to estimate a 3D volume for each 2D projection in real-time. However, since PCA only captures linear relationships between variables, it cannot accurately model the highly irregular, nonlinear dynamics of breathing motion that can change significantly from breath to breath. Deep neural networks have also been used to learn the corresponding 3D volumes directly from 2D projection data, but these methods operate solely in image space and do not provide motion data³. In Shen et al⁴., 3D volumetric images are predicted from 2D coronal slices using a neural network. However, this process requires a much larger neural network architecture and thus has greater memory requirements and produces output images much slower. The method described by Shen et al also cannot be used for efficient 2D-3D image registration and volumetric imaging is achieved only for certain imaging angles. ² Li R. Jia X. Lewis JH. et al. Real-time volumetric image reconstruction and 3D tumor localization based on a single x-ray projection image for lung cancer radiotherapy. Medical Physics. 2010;37(6Part1):2822-2826.; Lei Y. Tian Z. Wang T. et al. Deep learning-based real-time volumetric imaging for lung stereotactic body radiation therapy: a proof of concept study. Physics in Medicine & Biology. 2020;65(23):235003.³ Shen L. Zhao W. Xing L. Patient-specific reconstruction of volumetric computed tomography images from a single projection view via deep learning. Nature Biomedical Engineering. 2019;3(11):880-888.; Lei Y. Tian Z. Wang T. et al. Deep learning-based real-time volumetric imaging for lung stereotactic body radiation therapy: a proof of concept study. Physics in Medicine & Biology. 2020;65(23):235003.⁴ Shen L. Zhao W. Xing L. Patient-specific reconstruction of volumetric computed tomography images from a single projection view via deep learning. Nature Biomedical Engineering. 2019;3(11):880-888

Any discussion of the prior art throughout the specification should in no way be considered as an admission that such prior art is widely known or forms part of common general knowledge in the field.

### SUMMARY

### Problems to be Solved

It is an aim and objective of the present invention to provide an improved 2D/3D registration and volumetric imaging method and system in terms of reducing cost and reducing time to complete the process.

It is an aim and objective of the present invention to provide a 2D/3D registration and volumetric imaging method that can be implemented by a system in real time.

It is an aim and objective of the present invention to provide a 2D/3D registration and volumetric imaging method that can be used in an inexpensive motion management system that can be readily integrated into an existing clinical workflow without the need for additional equipment or training.

It is an aim and objective of the present invention to provide a 2D/3D registration and volumetric imaging method that can be used with gantry-based linear accelerators and on-board x-ray imaging.

It is an aim and objective of the present invention to provide a 2D/3D registration and volumetric imaging method that can be used across many imaging modalities (MRI, Ultrasound, X-ray etc)

It is an aim and objective of the present invention to provide a 2D/3D registration and volumetric imaging method that can be seamlessly integrated into the existing workflows and systems used for radiotherapy.

It is an object of the present invention to overcome or ameliorate at least one of the disadvantages of the prior art, or to provide a useful alternative.

### Means for Solving the Problem

According to a first aspect, the invention provides a system for image registration and volumetric imaging of a patient, the system comprising: a first interface adapted to receive 3D and/or 4D images of the patient; a second interface adapted to receive 2D images during a procedure carried out on the patient; and a processing unit wherein the processing unit is adapted to carry out a method comprising the steps of: acquiring a static 3D image or a dynamic 4D image of the patient prior to the procedure; if a dynamic 4D image is acquired, performing deformable image registration between a reference 3D image and each 3D image taken from the dynamic 4D image to produce a set of 3D deformation vector fields; if a static 3D image is acquired, applying known 3D deformation vector fields to the static 3D image acquired prior to the procedure, to produce a dynamic 4D image; projecting the acquired static 3D image or acquired dynamic 4D image to produce a set of corresponding 2D images; training a deep neural network using a moving 3D image, wherein the moving 3D image is derived from the acquired dynamic 4D image or the acquired static 3D image and the corresponding 2D images to produce 3D deformation vector fields and estimate fixed 3D images simultaneously; acquiring 2D images of the patient during the procedure carried out on the patient; generating real-time 2D to 3D image registration and volumetric imaging by inputting 2D images acquired during the procedure into the deep neural network.

Preferably, 2D images are acquired in real time during the procedure. Preferably, a 2D image registration can be used to estimate a 2D deformation vector field to an acquired fixed 2D image. Preferably, one or more fixed 2D images can be acquired at one or more angles around the patient. Moving 2D images can preferably be acquired by forward-projecting the updated 3D image at the same angles prior to treatment.

Preferably, the 3D deformation vector field and the 2D deformation vector field can be related by a mathematical mapping wherein the mathematical mapping can be learnable by the deep neural network.

Preferably, the motion of a structure in the 3D volumetric image is determined based on the fixed 2D images that are continuously acquired during the procedure so that the procedure is focussed on a patient's target organ while avoiding organs at risk. Preferably, the deep neural network can be used to estimate both 3D DVFs and fixed 3D images at a moment of interest based on fixed 2D images acquired at the moment of interest, prior moving 2D images and a prior updated moving 3D image.

Preferably, the system can access on-board imaging available on a standard linear accelerator.

The 3D volumetric image is preferably an image selected from the group consisting of a computed-tomography image, a magnetic resonance image, a positron emission tomography image, a synthetic image, and an X-ray image.

The 4D-CT images are preferably of the patient's respiratory system or abdominal area.

The network preferably comprises a computer equipped with a GPU to train and deploy the neural network, wherein the neural network is constructed, trained and tested using a programming language and a machine learning library.

According to a second aspect, the invention provides a method for image registration and volumetric imaging, the method comprising: acquiring a static 3D image or a dynamic 4D image of the patient prior to the procedure; if a dynamic 4D image is acquired, performing deformable image registration between a reference 3D image and each 3D image taken from the dynamic 4D image to produce a set of 3D deformation vector fields; if a static 3D image is acquired, applying known 3D deformation vector fields to the static 3D image acquired prior to the procedure, to produce a dynamic 4D image; projecting the acquired static 3D image or acquired dynamic 4D image to produce a set of corresponding 2D images; training a deep neural network using a moving 3D image, wherein the moving 3D image is derived from the acquired dynamic 4D image or the acquired static 3D image, and the corresponding 2D images to produce 3D deformation vector fields and estimated fixed 3D images simultaneously; acquiring 2D images of the patient during the procedure carried out on the patient; and generating real-time 2D to 3D image registration and volumetric imaging by inputting 2D images acquired during the procedure into the deep neural network.

The method preferably further comprises acquiring one or more fixed 2D images at given points in time, wherein the 2D images are acquired in real time during the procedure. Preferably, a 2D image registration can be used to estimate a 2D deformation vector field to an acquired fixed 2D image. Preferably, one or more fixed 2D images can be acquired at one or more angles around the patient and moving 2D images can be acquired by forward-projecting the updated 3D image at the same angles prior to treatment.

Preferably, the 3D deformation vector field and the 2D deformation vector field can be related by a mathematical mapping wherein the mathematical mapping can be learnable by the deep neural network, wherein the network comprises a computer equipped with a GPU to train and deploy the neural network, wherein the neural network is constructed, trained and tested using a programming language and a machine learning library.

The motion of a structure in the 3D volumetric image is preferably determined based on the fixed 2D images that are continuously acquired during the procedure so that the procedure is focussed on a patient's target organ while avoiding organs at risk.

The deep neural network can preferably be used to estimate both 3D DVFs and fixed 3D images at a moment of interest based on fixed 2D images acquired at the moment of interest, prior moving 2D images and a prior updated moving 3D image.

The 3D volumetric image is preferably an image selected from the group consisting of a computed-tomography image, a magnetic resonance image, a positron emission tomography image, a synthetic image, and an X-ray image, wherein the method can be carried out by accessing on-board imaging available on a standard linear accelerator.

In the context of the present invention, the words “comprise”, “comprising” and the like are to be construed in their inclusive, as opposed to their exclusive, sense, that is in the sense of “including, but not limited to”.

The invention is to be interpreted with reference to the at least one of the technical problems described or affiliated with the background art. The present aims to solve or ameliorate at least one of the technical problems and this may result in one or more advantageous effects as defined by this specification and described in detail with reference to the preferred embodiments of the present invention.

### BRIEF DESCRIPTION OF THE FIGURES

FIG. 1 discloses a pictographic representation of respiratory motion estimation and volumetric imaging formulated as a problem of domain-transform via manifold learning according to a preferred embodiment of the invention;

FIG. 2 discloses a clinical workflow for a preferred embodiment of the invention;

FIG. 3 discloses a pictographic representation of the architecture of a network used for a preferred embodiment of the invention;

FIG. 4a discloses supervised training of the network converged over the course of 50 epochs for a preferred embodiment of the invention.

FIG. 4b discloses unsupervised training of the network converged over the course of 50 epochs for a preferred embodiment of the invention.

FIG. 5 discloses supervised predicted and ground-truth target 3D deformation vector fields on seen data for a preferred embodiment of the invention.

FIG. 6 discloses unsupervised predicted and ground-truth target 3D deformation vector fields on seen data for a preferred embodiment of the invention.

FIG. 7 discloses supervised predicted and ground-truth target 3D deformation vector fields on unseen data for a preferred embodiment of the invention.

FIG. 8 discloses unsupervised predicted and ground-truth target 3D deformation vector fields on unseen data for a preferred embodiment of the invention.

### DESCRIPTION OF THE INVENTION

Preferred embodiments of the invention will now be described with reference to the accompanying drawings and non-limiting examples.

In this document, CT refers to computed tomography, CBCT refers to cone beam computed tomography, and DVF refers to deformation vector field.

As a medical treatment, radiotherapy is essential in alleviating the global burden of cancer but the deadliest cancers involve tumours in the thorax and abdomen, which move due to respiration. This constant and irregular motion necessitates the use of motion management to track tumour motion and minimise healthy tissue damage. Indeed, the majority of radiotherapy centres around the world wish to utilize real-time motion management during treatment. However, access to motion management technologies is limited by finances, human resources and machine capacity. The Applicants advantageously found that developing a patient-specific deep learning framework for use in the method of the present invention can predict respiratory motion and volumetric images from 2D images acquired during radiotherapy. The method and system of the present invention can be advantageously leveraged to access the on-board imaging available on all standard linear accelerators, and could facilitate real-time, cost-effective motion management for radiotherapy centres around the world. One important challenge for the use of on-board systems is that 2D imaging must be used to capture 3D motion. Indeed, there will always been information that is lost in projecting 3D information onto a 2D plane. For instance, tumours can come obscured at certain imaging angles, especially when aligned with high intensity structures such as bone.

In a preferred embodiment of image-guided radiotherapy, a moving 3D image is acquired prior to treatment. This image (the “planning day image”) is used to delineate the target object (typically a malignant tumour) as well as any nearby organs at risk (e.g. the heart, lungs, oesophagus, spine). In a preferred method of the invention, the motion of these delineated structures is determined based on fixed 2D images that are continuously acquired as patients are treated with radiotherapy so that the radiation is focussed on the target tumour while avoiding organs at risk.

Importantly, there is often several weeks between acquiring this moving 3D “planning day” image and the treatment day on which the fixed 2D images are acquired. There are two pieces of hardware for are typically required for this process: a pre-treatment scanner and a desktop computer. Immediately prior to treatment, a pre-treatment “treatment day” scan, such as cone beam computed tomography (CBCT), is performed. This can be considered as a quick, low quality computer tomography (CT) scan that is used by a radiographer to determine how the relevant anatomical structures have shifted between the planning CT scan to the treatment day. Image registration is then performed between the images obtained in the “planning day” CT scan and the “treatment day” CBCT scan to produce an “updated treatment day” CT scan that accounts for any anatomical changes in the interim. It is advantageous to avoid performing another CT scan on the day of treatment. A CT scan takes much longer to perform than a quick CBCT scan and a CT scan exposes the patient to a much higher level of radiation than a CBCT scan. Therefore, in the method of the present invention, a pre-treatment CBCT scan can be performed to produce a low-quality “treatment day” 3D image, which is then used to update the moving 3D image obtained on the “planning day” via 3D image registration. It was surprisingly found that moving 2D images can be produced by acquiring 2D projections through the “updated treatment day” 3D image. 2D image registration can be used to estimate a 2D deformation vector field to the fixed 2D images being acquired during treatment. In particular, during image-guided radiotherapy on a standard linear accelerator, fixed 2D images are acquired at various angles as the gantry rotates around a stationary patient and moving 2D images can be acquired by forward-projecting the updated 3D image at the same angles prior to treatment.

However, the present invention is adapted to derive the 3D motion of structures delineated on the updated moving 3D image. The second aspect of the present invention provides a method where the desired 3D deformation vector field and the aforementioned 2D deformation vector field (DVF) are derived by a mathematical mapping carried out by the derive of the present invention. This mapping is adapted to be updated by an artificial intelligence system. In one embodiment, the mapping is adapted to be updated by an artificial intelligence system during intra-intervention procedures. The system of the present invention is adapted to generate a desired 3D DVF and applying it to the updated 3D image. This will then provide a fixed 3D image at the moment of interest. In other words, an artificial intelligence system can be used to estimate both 3D DVFs and fixed 3D images at the moment of interest given fixed 2D images acquired at the moment of interest, prior moving 2D images and a prior updated moving 3D image.

### Method

In the preferred embodiment of the invention, the present invention provides a method and system for efficient 2D-3D image registration and volumetric imaging using a deep neural network for x-ray guided radiotherapy. In this context, the system consists of a first interface, such as CT scanner to acquire 3D volumetric images prior to treatment, a second interface, such as a standard radiotherapy system to acquire 2D images during treatment and a device equipped with a processing unit adapted to conduct machine learning, including the train and deploy a neural network. For the experiments described herein, a deep neural network was constructed, trained and tested using the Python programming language and the Pytorch machine learning library. All imaging data was produced using the MATLAB programming language. In one embodiment, the processing unit is a vector processor, a GPU, or a processor that contains on-chip acceleration for AI inferencing.

A key advantage of the present invention is the registration of images taken during treatment to those acquired during planning. In existing methods, the clinical team has no information regarding whether the contouring of organs/tumours/etc and determining radiation dose/distribution/treatment angles/etc evaluated on the planning day is still relevant on the treatment day. The method and system of the present invention essentially updates the information obtained on the planning day so that this information is still relevant during treatment without requiring another full CT scan on the treatment day.

### 2.1 Workflow

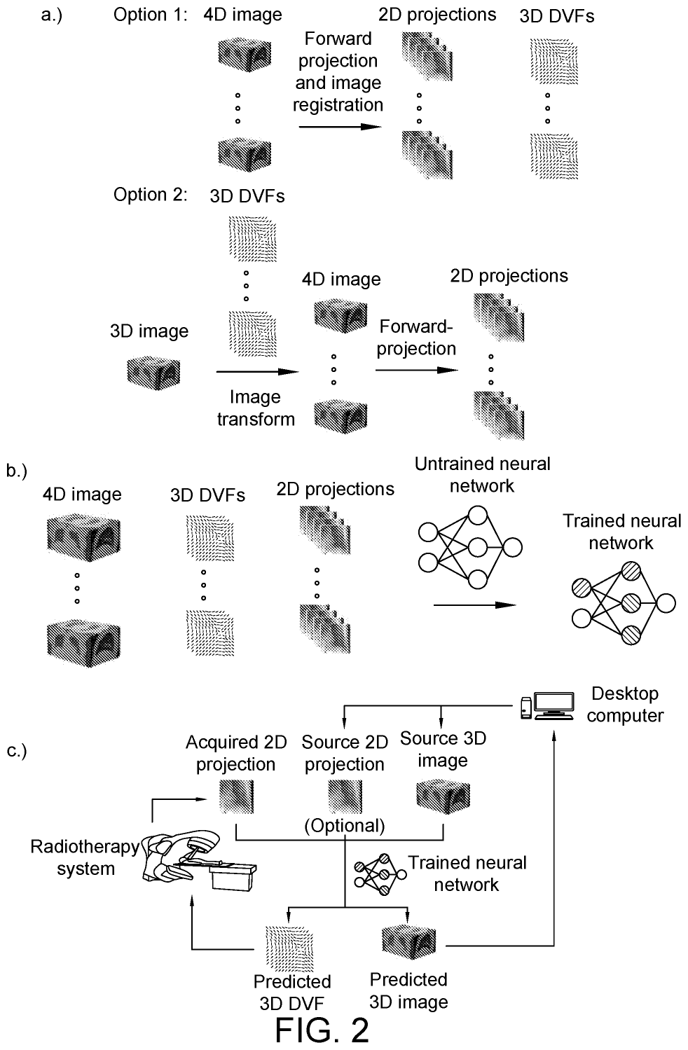

In one embodiment, the method carried out by the system of the present invention comprises 6 steps:
(a) acquire a respiratory-correlated 4D-CT of a patient prior to treatment. A 4D-CT produces a number (e.g. 10) of distinct 3D images;
   Alternatively, a conventional CT, MRI or ultrasound can be used to obtain a 3D reconstructed image and motion fields can be applied to the image to generate additional 3D images for training.
(b) project each of the distinct 3D volumetric images via the Radon transform to produce a set of corresponding 2D images;
(c) As shown in Option 1 of FIG. 2a, if a 4D image is acquired, perform deformable image registration between a reference 3D image and each 3D image of the 4D-CT to produce a set of 3D deformation vector fields;
(d) As shown in Option 2 of FIG. 2a, if a static 3D image is acquired, apply known 3D deformation vector fields to the static 3D image acquired prior to the procedure, to produce a dynamic 4D image;
(e) projecting the acquired static 3D image or acquired dynamic 4D image to produce a set of corresponding 2D images;
(f) train a deep neural network using the 3D volumetric images and corresponding 2D images to produce 3D deformation vector fields and deformed 3D volumetric images simultaneously;
(g) acquire 2D images of the patient during treatment;
(h) use the 2D images acquired during treatment and corresponding 2D images from a reference 3D volumetric image as input to the neural network to achieve efficient 2D-3D image registration and volumetric imaging. This reference 3D image is typically selected as one of the 10 3D images in step (a). In a particularly preferred embodiment, the peak-exhale image of the patient's lung is taken as the reference image, as this usually contains the fewest motion artefacts (blurring, etc) and is therefore used by the clinical team for treatment planning.

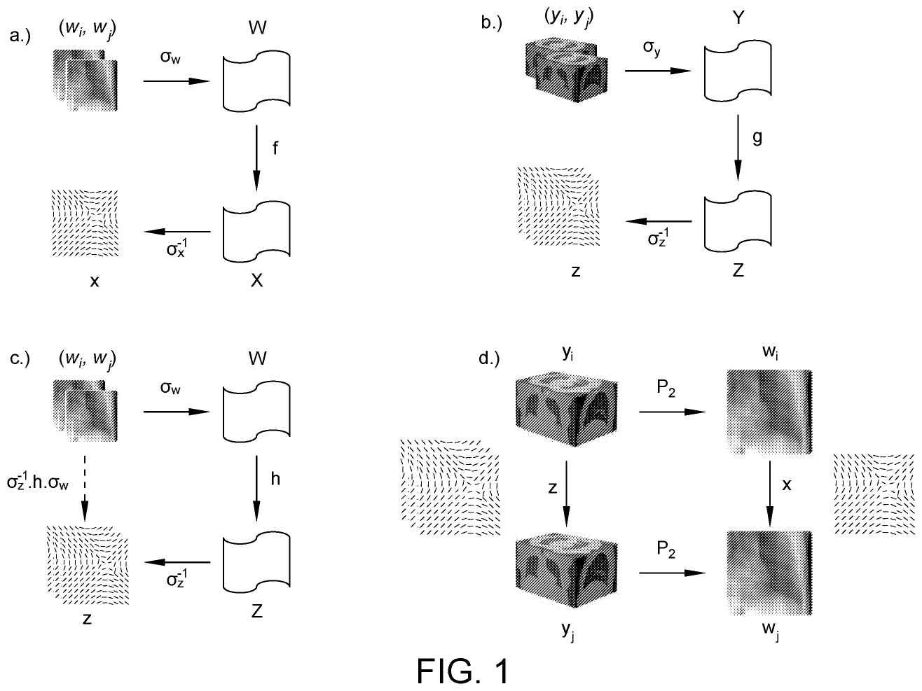

Referring to FIG. 1, respiratory motion estimation and volumetric imaging is formulated as a problem of domain-transform via manifold learning. In step a., 2D image registration is formulated as mapping between manifold representations of 2D projection pair (wᵢ,wⱼ) and 2D deformation vector field (DVF) x. In step b., 3D image registration is formulated as mapping between manifold representations of 3D image pair (yᵢ,yⱼ) and 3D DVF z. In step c., 2D to 3D image registration is formulated as mapping between manifold representations of 2D projection pair (wᵢ,wⱼ) and 3D DVF z. In step d., 3D image pair (yᵢ,yⱼ) is related by a 3D DVF z, 2D projection pair (wᵢ, wⱼ) is related by a 2D DVF x and 2D projections are related to 3D images by projection operator P₂.

In the present invention, there is provided a patient-specific deep learning framework for real-time respiratory motion estimation and volumetric imaging. Referring to FIG. 1, W and X represent manifolds over a training corpus of 2D projection pairs and 2D DVFs, respectively, such that the function σ_{z} that maps every 2D projection pair (wᵢ,wⱼ) onto W and the function ox that maps every 2D DVF x onto X. Similarly, Y and Z represent manifolds over a corpus of 3D image pairs and 3D DVFs, respectively, such that σ_{y} maps every 3D image pair (yᵢ, yⱼ) onto Y and σ_{z} that maps every 3D DVF z onto Z. Supposing functions f and g map from image space to DVF space in the 2D and 3D cases respectively. Indeed, this can be viewed as a geometric learning approach to the recent use of neural networks in medical image registration. According to the method of the present invention, if every 3D image pair differs only by respiratory motion for a given patient, then there exists a third function h that maps from the manifold over 2D projection pairs W to the manifold over 3D DVFs Z. Further, the composite transformation z=σ_{z}⁻¹·h·σ_{w} (wᵢ,wⱼ) can advantageously be learned by a deep neural network.

The Applicant of the present invention found that the existence of this third function is based on the notion that 3D DVFs do not differ from 2D DVFs arbitrarily but instead obey distinct biomechanical patterns. For instance, inhalation involves simultaneous inferior and anterior motion of the diaphragm such that an inferior shift in the coronal plane corresponds to an anterior shift in the sagittal plane. Lateral expansion of the ribs in the coronal plane is associated with dorsiventral expansion in the axial plane etc. In other words, there is an “allowed” set of candidate 3D DVFs given the corresponding 2D DVFs that depends on the specific breathing patterns of each patient. Similarly, 3D image pair (yᵢ,yⱼ) cannot differ arbitrarily from 2D projection pair (wᵢ,wⱼ) but instead reflects the unique anatomy of each patient. The system and method of the present invention advantageously leverages these constraints to achieve real-time respiratory motion estimation and volumetric imaging in a patient-specific manner given the images available in existing workflows for cancer radiotherapy.

### 2.2 Data

Referring to FIG. 2, a training corpus of 3D images, 2D projections and 3D DVFs was created using data from two lung cancer patients. 3D images were acquired by performing a CT for each patient and retrospectively sorting the resulting slices by a respiratory signal, also known as a 4D-CT. For instance, y₁ is the 3D image corresponding to peak-inhalation, y₂ is that corresponding to 20% exhale, etc. Deformable image registration was performed between a reference 3D image y_{ref} and every 3D image of the 4D-CT to yield 10 3D DVFs (including the identity mapping between y_{ref} and itself), which are used as zₜᵣᵤₑ. y_{ref} was selected as the peak-exhalation image as this typically contains the fewest motion artefacts and is therefore used by the clinical team for treatment planning.

Each 3D image was also forward-projected to generate an array of 2D projections. Since the angles at which the patient will be imaged on the treatment day are typically known beforehand, this set of 2D projections represents the 2D appearance of the unique anatomy of the patient at the desired angles. During training, a 2D projection from a random angle for a random 3D image y_{target} was taken as input as well as the 2D projection from the same angle as y_{ref}. The method of the present invention then uses each 2D projection pair to estimate a 3D DVF z_{pred} and network optimization proceeded by minimising the difference between z_{pred} and zₜᵣᵤₑ. The estimated DVF z_{pred} was also passed through a spatial transformation network to warp y_{ref}, enabling volumetric imaging. Monte Carlo simulation was used to create 2D projections without scatter for Patient 1 and with scatter for Patient 2 to determine whether network performance differed under conditions typically encountered in radiotherapy.

As can be seen in FIG. 2, a clinical workflow for a preferred embodiment of the method of the present invention comprises the following steps:
a. 4D-CT is processed by a desktop or laptop computer to produce 2D projections and 3D DVFs;
b. Training proceeds by taking 2D projections as input and comparing the predicted 3D DVFs to pre-computed 3D DVFs;
c. 2D to 3D image registration and volumetric imaging proceeds by using acquired 2D projections from the radiotherapy system as well source 2D projections and 3D images from a desktop computer as input.

The trained network then predicts 3D DVFs which can be used for real-time beam adaptation and 3D images which can be viewed during treatment.

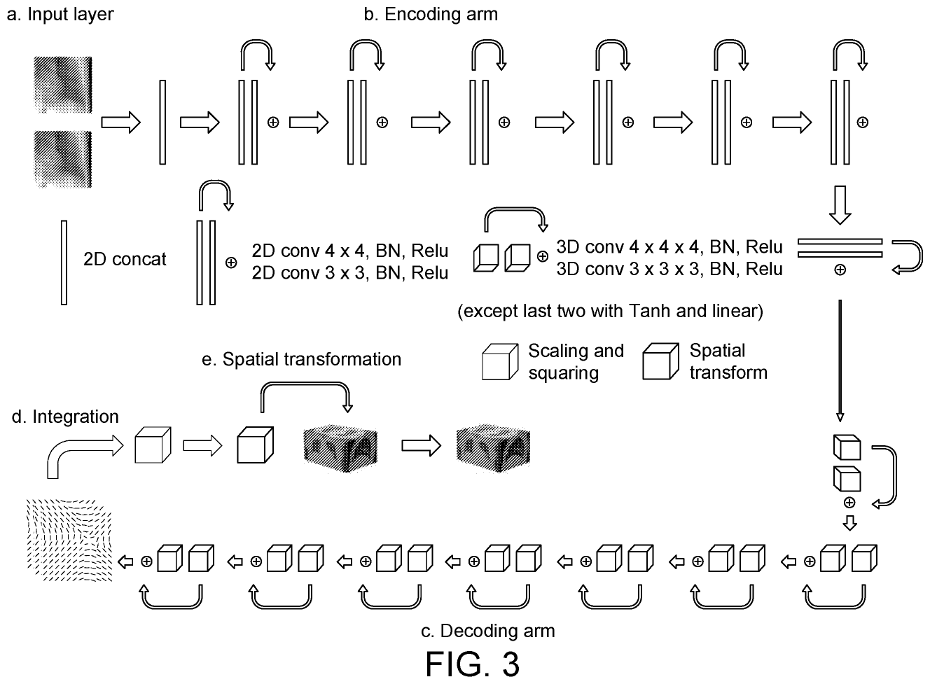

Referring to FIG. 3, an example of the architecture of programming of the system of the present invention can be described as follows. Other programming architecture may be possible depending on the final application of the system and method:
a. The input layer concatenates 2D projection pairs;
b. The encoding arm consists of n repeating residual blocks (where 2D projection size=2ⁿ×2ⁿ) followed by reshaping to a 3D tensor;
c. The decoding arm consists of n repeating residual blocks followed by two additional convolutions at the desired 3D DVF size (3×2ⁿ×2ⁿ×2ⁿ);
d. Optional integration layers can be included to encourage diffeomorphic transformations;
e. Output 3D images are produced by passing output 3D DVFs and source 3D images through spatial transformation layers.

In one preferred embodiment of the present invention, imaging data was acquired using scans from a Sparse-view reconstruction (SPARE) challenge. In this challenge, 1-min cone beam computed tomography (CBCT) scans were simulated using 4D-CT volumes for patients with locally advanced non-small-cell lung cancer receiving 3D conformal radiotherapy. 4D-CT volumes used to simulate planning were acquired on a different day to those used to simulate treatment. For the planning day data, the 4D-CT supplied 10 3D volumetric images, each of which was projected to produce 2D images at 680 angles for one 360° revolution. This yielded 6800 volume-projection pairs with a 90:10 training:validation split—i.e. 6120 images for training and 680 images for validation. For the treatment day data, respiratory motion was simulation by converting real-time position management (Varian Medical Systems, Palo Alto, US) traces into respiratory phases and acquiring projections for the corresponding 4D-CT volumes at 680 angles for one 360° revolution. This yielded 680 volume-projection pairs for testing. All projections were simulated for a 120 kVp beam with a pulse length of 20 ms going through a half-fan bowtie filter.

Scatter and noise profiles were generated via Monte Carlo simulation to produce distinct simulation types for each patient:

Patient 1 (without scatter): primary and noise signals at 40 mA tube current

Patient 2 (with scatter): primary, scatter, and noise signals at 40 mA tube current

These simulation types enabled determination of whether performance of the neural network differed under imaging conditions commonly encountered in radiotherapy

Every 2D projected image was initially generated with pixel sizes of 0.776 mm×0.776 mm and dimensions 512×384. However, the 2D images were down-sampled to 128×128 due to memory constraints. Similarly, every 3D volumetric image was initially generated voxel sizes of 1 mm×1 mm×1 mm and dimensions 450×220×450 but was reshaped to 512×256×512 using bicubic interpolation and down-sampled to 128×128×128. Pixel intensities for all images were normalised between 0 and 1. Lung masks were delineated by a clinician for each patient on the peak-exhalation 4D-CT. These lung masks were used to generate masks of the thorax by generating a convex hull to include both lungs, expanding the resulting hull by binary dilation to include the ribs and extension inferiorly to the bottom of the image. Deformable image registration between the peak-exhalation 4D-CT and every other image of the 4D-CT for the simulated planning and treatment days was performed using the Elastix toolkit.

### 2.3 Neural Network Architecture

A deep neural network (DNN) is an artificial neural network (ANN) with multiple layers between the input and output layers. There are different types of neural networks but they always consist of the same components: neurons, synapses, weights, biases, and functions. Each mathematical manipulation in a neural network as such is considered to be a “layer” and complex DNN have many layers, hence the name “deep” networks.

DNNs are usually feedforward networks in which data flows from the input layer to the output layer without looping back. Initially, the DNN creates a map of virtual neurons and assigns random numerical values, or “weights”, to connections between them. The weights and inputs are multiplied and return an output between 0 and 1. If the network does not accurately recognize a particular pattern, an algorithm would adjust the weights. The algorithm can adjust to give certain parameters greater influence until the correct mathematical manipulation is found to fully process the data.

The neural network architecture for this embodiment consists of 4 components: (1) an encoding arm (2) a decoding arm (3) integration layers (4) a spatial transformation module. This is illustrated in FIG. 2. The input layer concatenates 2D projection pairs and the encoding arm serves to produce a latent, low-dimensional representation of the 2D DVF between 2D projection pairs. Conversely, the decoding arm serves to map from this latent, low-dimensional representation to the 3D DVF between 3D image pairs. The integration layers serve to compute the integral of the output from the final layer of the decoding arm. This is to encourage diffeomorphic transformations. Lastly, the spatial transformation module serves to deform the source 3D image using the output 3D DVF of the neural network, enabling volumetric imaging.

In this embodiment, 2D projection pairs are concatenated to produce an input tensor of dimensions 2×128×128, where the first number indicates the number of channels while the second and third indicate image size. This is fed into the encoding arm of the neural network, which consists of n=7 (since image size=128×128=2⁷×2⁷) repeating residual blocks where each input tensor is convolved with a kernel of size 4×4 with stride 2 and padding 1, followed by a kernel of size 3×3 with stride 1 and padding 1 and batch normalization. This repeating pattern of convolutions yields output images at half the dimension of the original inputs and the number of output channels is chosen as double that of the original input. These values represent a balance between processing fine and coarse motion. As the patient is imaged, there will be coarse motion due to translation by the diaphragm as well as fine motion due to the deformation of certain structures, intercostal muscles, etc. Both are considered to be important to get a holistic picture of internal anatomical motion.

Hence, the first residual block produces an output tensor of dimension 4×64×64, the next residual block produces an output tensor of dimension 8×32×32 and so on until the final block of the encoding arm with output dimensions 256×1×1. Importantly, the number of residual blocks is determined intrinsically by the size of the input images such that larger images with detailed DVFs are processed by larger networks. Residual blocks are used because a deep neural network is required, introducing more layers (vanishing gradients and degradation) and residual blocks comprise connections that can be used to address such problems.

In a reciprocal manner to that of the encoding arm, the decoding arm also consists of n=7 repeating residual blocks. However, each block performs transpose convolution with a kernel of size 4×4×4 with stride 2 and padding 1, followed by a kernel of size 3×3×3 with stride 1 and padding 1 and batch normalization. These block values are examples of a preferred embodiment only and can vary depending on the final application according to the invention. Two additional convolutions are then performed at the desired output image size of 128×128×128 with kernels of size 3×3×3 with stride 1 and padding 1, and the number of channels is chosen to produce a final output tensor of dimensions 3×128×128×128. Every convolution, transpose convolution and fully connected layer uses Rectified Linear Activation Function (ReLu) activation, except the penultimate and final layers which use Tanh and linear activation respectively.

The output of the decoding arm is then fed through scaling and squaring layers (step size=10,) which efficiently integrate the corresponding tensor. This process yields a 3D DVF, which is then fed into a spatial transform module along with the source 3D image to produce a warped 3D image.

The Applicants surprisingly found that all neural networks could be trained on a commercially available laptop computer. (Lambda Tensorbook (2020 model)—Machine Learning, Deep Learning, Data Science Laptop: Intel Core i7-10875H 8 Core, NVIDIA RTX 2080 Super Max-Q 8 GB, 15.6″ 1080p, 64 GB RAM, 1 TB NVMe SSD, Thunderbolt 3). In a treatment environment, the workflow could be carried out using the available desktop or laptop computers or server in the radiotherapy bunker.

### 2.4 Neural Network Training

The neural network can be trained in a supervised or unsupervised manner. For supervised training, the loss function for training was defined MSE (zₜᵣᵤₑ, z_{pred}), where MSE is mean squared error, zₜᵣᵤₑ is the true 3D DVF and z_{pred} is the predicted 3D DVF. To encourage the network to focus on learning thoracic motion, this loss computed only within a mask of the thorax. The neural network was trained using the Adam learning algorithm with learning rate 1×10⁻⁵ and batch size 2 for 50 epochs to ensure convergence. In other words, the network is trained by maximising similarity between true and predicted 3D motion.

For unsupervised training, the loss function was defined as MSE(x−y)+α grad(v), where the first term represents a similarity metric between the true target 3D volumetric image x and the deformed 3D volumetric image y while the second term represents a smoothness metric for the predicted target 3D deformation vector field v. In this embodiment, this second term was chosen as the gradient of the predicted 3D deformation vector field but other smoothness metrics such as the binding energy or L1/L2-norm could be used. This term is necessary in the unsupervised regime to regularise the solution space. Here, we choose α=1×10⁻⁵.

Once trained, the network was tested using simulated treatment images. Importantly, these images and the corresponding DVFs were unseen during training. To evaluate DVF accuracy, mean 3D error (in voxels) between the ground-truth and predicted 3D DVFs in the thorax and tumors were recorded. To evaluate image accuracy root-mean-squared error (RMSE), mean average error (MAE), structural similarity (SSIM) and peak signal-to-noise ratio (PSNR) in pixel intensities between the ground-truth and predicted 3D volumetric images were recorded. Lastly, to evaluate network efficiency, mean inference time and the number of trainable parameters were recorded.

### 3. Results

### 3.1 Training of the Neural Network

It is understood that unsupervised training refers to training without access to ground-truth data, while supervised training refers to training with access to ground-truth data. More specifically, the task for the neural network here is 2D/3D registration so the desired output is a 3D deformation vector field. When the network is trained in an unsupervised manner, the accuracy of the predicted deformation vector field is assessed by using it to deform a reference 3D image and assessing how different this warped image is to a target image. Conversely, when a neural network is trained in a supervised manner, the predicted 3D deformation vector field is directly compared to the target vector field

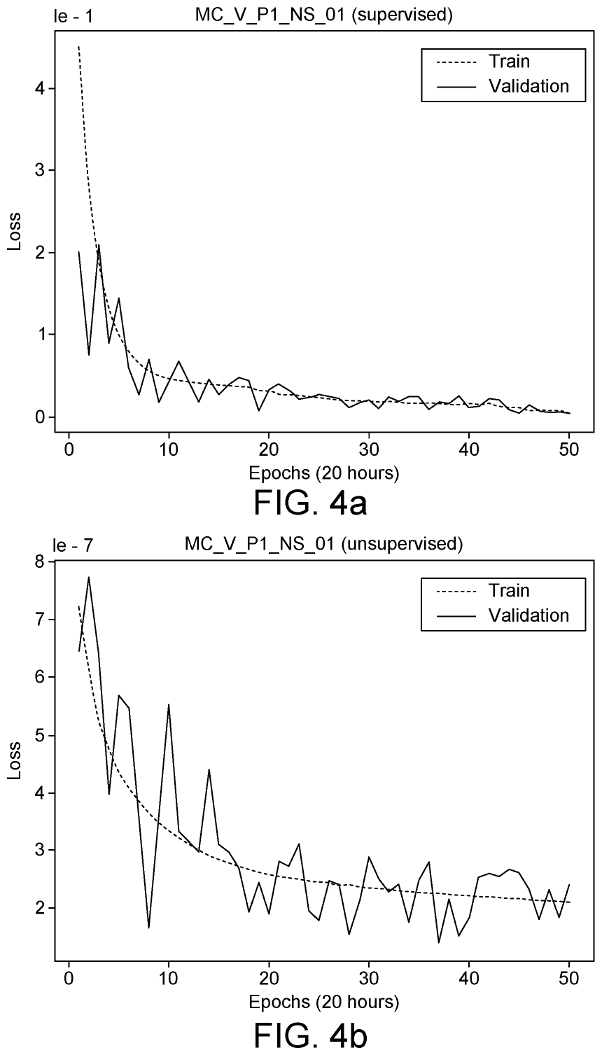

FIG. 4a discloses supervised training of the network converged over the course of 50 epochs.

In the unsupervised case, as shown in FIG. 4b, the training is still converged overall. However much greater fluctuation of the validation data was observed.

Importantly, both networks took approximately 20 hours to train. Once trained they were able to produce 3D deformation vector fields and volumetric images in 50 ms. This suggests the possibility of real-time implementation with this method.

### 3.2 Performance on Seen Data

In the context of the present invention, seen data is data that is used during training of the neural network. Unseen data is data that is not used during training of the neural network.

In the context of the preferred embodiment of the present invention, the neural network is preferably trained on pre-treatment data and validated on intra-treatment data. Hence, intra-treatment data is preferably unseen during training of the neural networks of the preferred embodiments of the invention.

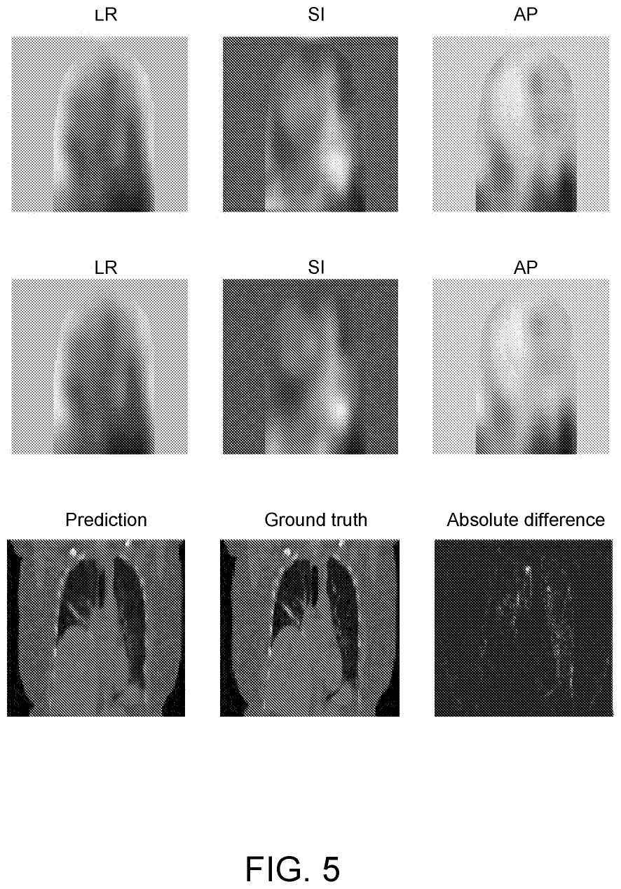

As shown in FIG. 5, the supervised network performed excellently on seen data (below) both in terms of the predicted target 3D deformation vector fields and 3D volumetric images. The top row is the predicted target 3D deformation vector field according to the neural network, the middle row is the ground-truth 3D deformation vector field from an Elastix registration and the last row compares predicted and ground-truth volumetric images.

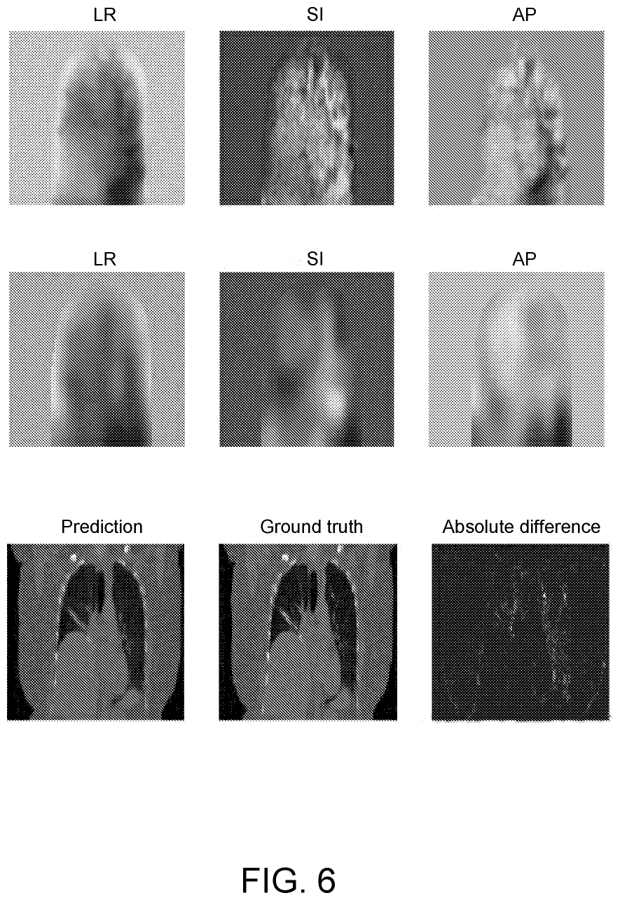

As shown in FIG. 6, the unsupervised network performed similarly to the supervised network on seen data. However, the target 3D deformation vector fields appear significantly different from the Elastix registration. The top row is the predicted target 3D deformation vector field according to the neural network, the middle row is the ground-truth 3D deformation vector field from an Elastix registration and the last row compares predicted and ground-truth volumetric images.

### 3.3 Performance on Unseen Data

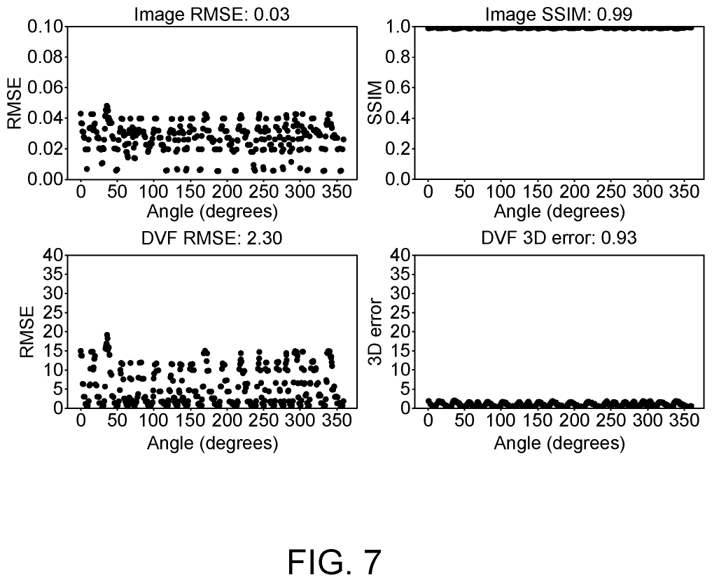

As shown in FIG. 7, the supervised network performed well on unseen data both in terms of images and deformation vector fields (below). There was some fluctuation in errors due to patient breathing, as expected the network performed best when the target volume was closest to the source volume and worse when they were farthest (peak-exhale and peak-inhale, respectively).

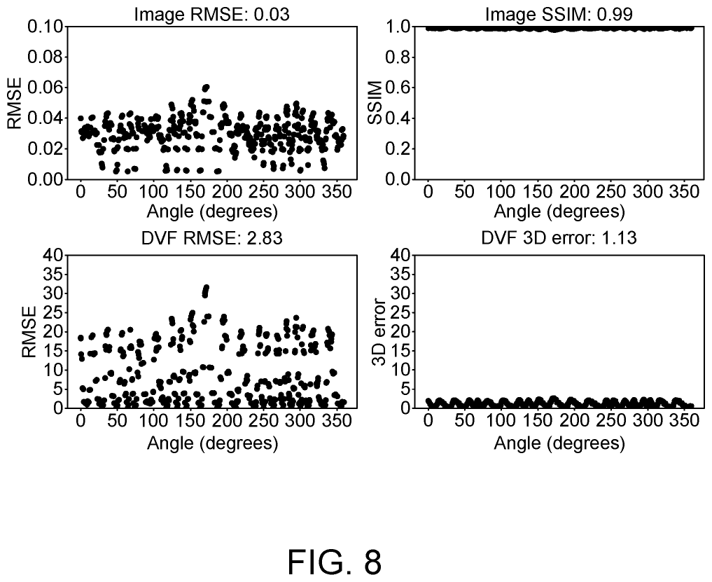

As shown in FIG. 8, the unsupervised network performed very similarly, with slightly greater errors in the DVF.

The performance of these networks coupled with their fast training and inference times, suggests that they could hold significant advancement in image-guided radiotherapy.

It can be shown that the present invention addresses the task of volumetric imaging specifically in the context of images that differ by respiratory motion while existing prior art methods consider volumetric imaging more broadly. The method and system of the present invention advantageously simultaneously predict both respiratory motion and volumetric images. This motion data is crucial for real-time motion management during radiotherapy and can also be used in other contexts, such as disease progression. Advantageously, the method and system of the present invention employ a much smaller network despite producing more information. Indeed, for the same image size, the system and method of the present invention require the storage of 50-fold fewer trainable parameters (10 million vs 500 million), thereby drastically decreasing memory requirements. The lightweight network of the present invention also performed inference approximately 10 times faster (50 ms vs 500 ms), suggesting the possibility of real-time implementation. Existing prior art methods are confined to producing volumetric images for single projections acquired at one angle, while the method and system of the present invention can be employed for 2D to 3D image registration and volumetric imaging at any angle. The method of the present invention requires a source 3D image for volumetric imaging while existing prior art methods do not. However, such images are available in the existing clinical workflows for cancer radiotherapy and allow the method of the present invention to adapt to anatomical changes between planning and treatment.

The method of the present invention can surprisingly continuously predict 3D tumour motion with mean errors of 0.1±0.5, −0.6±0.8, and 0.0±0.2 mm along the left-right, superior-inferior, and anterior-posterior axes respectively. also predicted 3D thoracoabdominal motion with mean errors of −0.1±0.3, −0.1±0.6, and −0.2±0.2 mm respectively. Moreover, volumetric imaging was achieved with mean average error 0.0003, root-mean-squared error 0.0007, structural similarity 1.0 and peak-signal-to-noise ratio 65.8. The results of this study demonstrate the possibility of achieving 3D motion estimation and volumetric imaging during lung cancer radiotherapy.

The present invention demonstrates a patient-specific deep learning framework that leverages the non-linear mathematics of manifolds and neural networks to achieve 3D motion estimation and volumetric imaging in a single shot. Further, proof-of-principle for this framework has been provided in the context of lung cancer radiotherapy.

A key motivator for the present invention is to understand how insights from the labour-intensive tasks of segmentation and treatment planning should be updated during treatment. With this in mind, the system and method of the present invention trains in a patient-specific manner using only data acquired on the planning day. To validate this method, a deep neural network was trained and tested on imaging data acquired on two separate days. During each forward-pass, the neural network first concatenates acquired and reference 2D images. This 2D image pair is then fed into an encoding arm, which can be thought of as generating a latent low-dimensional representation of the key features in 2D image space. This low-dimensional feature map is then reshaped to a 3D tensor for processing by a decoding arm to produce a 3D DVF. The Applicant included the additional constraint that the underlying manifolds must be differentiable and therefore that the desired transformations be diffeomorphic. (It should be noted that the diffeomorphic constraint was included optionally to demonstrate the possibility of such mappings but there is nothing in the mathematics of the present invention that requires such a constraint.) This constraint is imposed implicitly by using scaling and squaring layers to efficiently integrate the output of the decoding arm. The resulting 3D DVF is passed through spatial transformation layers along with a reference 3D image to produce a predicted 3D image (FIG. 3).

Estimating 3D motion from 2D images is a challenging task that has broad implications for image-guided interventions. The results of found by the Applicant using the system and method of the present invention suggest that this task can be made computationally tractable with an appropriate deep learning framework. In particular, the system and method of the present invention is demonstrated to achieve 3D motion estimation and volumetric imaging for the treatment site that experiences arguably the most motion of any site in radiotherapy: the lung. Moreover, motion estimation is further complicated in the context of image-guided radiotherapy by the fact that the gantry continuously rotates around the patient during image acquisition. Despite these challenges, the system and method of the present invention was able to continuously track tumour motion, specifically, and thoracoabdominal motion, more generally, to submillimetre accuracy with minimal differences across imaging angles.

Overfitting is a perennial challenge in machine learning that occurs when a large number of parameters are optimized to fit seen data but do not generalize well to unseen data. The system and method of the present invention addresses this challenge by training neural networks in a patient-specific manner. The core idea behind the present invention is that the problem of mapping from 2D images to 3D motion can be solved by learning manifold representations that reflect the specific biomechanics of the patient-of-interest. This approach lies in stark contrast to traditional machine learning in which large and varied data are used across a multitude of different patients. Indeed, the manifolds learned by the present invention provide implicit constraints on the registration task that reflect the specific anatomy of each patient and therefore should not be used across different patients. In other words, the method of the present invention leverages the accuracy and specificity of optimizing over a large number of parameters for a particular patient while avoiding the issue of generalization by never using the same parameters across different patients. In image-guided radiotherapy, training data can be produced abundantly for this purpose by forward-projecting 3D images acquired during pre-treatment scans. In the present invention, a 4D-CT was acquired for each patient yielding 10 3D images that were then each projected at 680 different angles, yielding almost 7000 training examples.

Deep neural networks have previously been used to map 2D x-ray projections to 3D computed tomography images without predicting 3D motion. Once the desired 3D images are produced, they can subsequently be used to estimate motion via image registration. However, motion is constrained in ways that images are not. By mapping to a constrained solution space, the present invention is able to continuously estimate respiratory-induced motion despite changing imaging angles. This flexibility is essential in the context of interventional and diagnostic procedures where images are acquired at many different angles. In contrast, previous volumetric imaging methods required the training of a new network on a different dataset for each imaging angle. Additionally, despite the significant flexibility of the system and method, the present invention has been surprisingly found to employ a much smaller network than that of existing methods. Indeed, for the same image size, the framework of the present invention requires the storage of 50-fold fewer trainable parameters (1×10⁷ vs 5×10⁸), thereby drastically decreasing memory requirements. The lightweight network of the present invention also performed inference in only 50 milliseconds. Moreover, existing volumetric imaging techniques were validated on “clean” digitally reconstructed radiographs, while the networks in this paper were trained and tested on scatter-and noise-corrupted images that reflect imaging conditions typically encountered in clinical scenarios.

In one embodiment of the invention, 2D-3D registration occurs by forward-projecting a reference 3D image to produce a 2D image that is then fed into the neural network with an acquired 2D image. In an alternative embodiment, an acquired 2D image and reference 3D image can be used, without the forward-projection step.

Although the invention has been described with reference to specific examples, it will be appreciated by those skilled in the art that the invention may be embodied in many other forms, in keeping with the broad principles and the spirit of the invention described herein.

The present invention and the described preferred embodiments specifically include at least one feature that is industrial applicable.

1. A system for image registration and volumetric imaging of a patient, the system comprising:
a first interface adapted to receive 3D and/or 4D images of the patient:
a second interface adapted to receive 2D images during a procedure carried out on the patient; and
a processing unit wherein the processing unit is adapted to carry out a method comprising the steps of:
acquiring a static 3D image or a dynamic 4D image of the patient prior to the procedure;
if a dynamic 4D image is acquired, performing deformable image registration between a reference 3D image and each 3D image taken from the dynamic 4D image to produce a set of 3D deformation vector fields;
if a static 3D image is acquired, applying known 3D deformation vector fields to the static 3D image acquired prior to the procedure, to produce a dynamic 4D image;
projecting the acquired static 3D image or acquired dynamic 4D image to produce a set of corresponding 2D images;
training a deep neural network using a moving 3D image, wherein the moving 3D image is derived from the acquired dynamic 4D image or the acquired static 3D image, and the corresponding 2D images to produce 3D deformation vector fields and estimated fixed 3D images simultaneously;
acquiring 2D images of the patient during the procedure carried out on the patient; and
generating real-time 2D to 3D image registration and volumetric imaging by inputting 2D images acquired during the procedure into the deep neural network.

2. The system of claim 1 wherein 2D images are acquired in real time during the procedure.

3. The system of claim 1 wherein a 2D image registration can be used to estimate a 2D deformation vector field to an acquired fixed 2D image.

4. The system of claim 3 wherein one or more fixed 2D images can be acquired at one or more angles around the patient.

5. The system of claim 4 wherein moving 2D images can be acquired by forward-projecting the updated 3D image at the same angles prior to treatment.

6. The system of claim 3 wherein the 3D deformation vector field and the 2D deformation vector field can be related by a mathematical mapping wherein the mathematical mapping can be learnable by the deep neural network.

7. The system of claim 3 wherein the motion of a structure in the 3D volumetric image is determined based on the fixed 2D images that are continuously acquired during the procedure so that the procedure is focused on a patient's target organ while avoiding organs at risk.

8. The system of claim 6 wherein the deep neural network can be used to estimate both 3D DVFs and fixed 3D images at a moment of interest based on fixed 2D images acquired at the moment of interest, prior moving 2D images and a prior updated moving 3D image.

9. The system of claim 1 wherein the system can access on-board imaging available on a standard linear accelerator.

10. The system of claim 1 wherein the 3D volumetric image is an image selected from the group consisting of a computed-tomography image, a magnetic resonance image, a positron emission tomography image, a synthetic image, and an X-ray image.

11. The system of claim 1 wherein the 4D-CT images are of the patient's respiratory system or abdominal area.

12. The system of claim 1 wherein the network comprises a computer equipped with a GPU to train and deploy the neural network, wherein the neural network is constructed, trained and tested using a programming language and a machine learning library.

13. A method for image registration and volumetric imaging, the method comprising:
acquiring a static 3D image or a dynamic 4D image of the patient prior to the procedure;
if a dynamic 4D image is acquired, performing deformable image registration between a reference 3D image and each 3D image taken from the dynamic 4D image to produce a set of 3D deformation vector fields;
if a static 3D image is acquired, applying known 3D deformation vector fields to the static 3D image acquired prior to the procedure, to produce a dynamic 4D image;
projecting the acquired static 3D image or acquired dynamic 4D image to produce a set of corresponding 2D images;
training a deep neural network using a moving 3D image, wherein the moving 3D image is derived from the acquired dynamic 4D image or the acquired static 3D image, and the corresponding 2D images to produce 3D deformation vector fields and estimated fixed 3D images simultaneously;
acquiring 2D images of the patient during the procedure carried out on the patient; and
generating real-time 2D to 3D image registration and volumetric imaging by inputting 2D images acquired during the procedure into the deep neural network.

14. The method of claim 13 further comprising acquiring one or more fixed 2D images at given points in time, wherein the 2D images are acquired in real time during the procedure.

15. The method of claim 14 wherein a 2D image registration can be used to estimate a 2D deformation vector field to an acquired fixed 2D image.

16. The system of claim 15 wherein one or more fixed 2D images can be acquired at one or more angles around the patient and moving 2D images can be acquired by forward-projecting the updated 3D image at the same angles prior to treatment.

17. The method of claim 15 wherein the 3D deformation vector field and the 2D deformation vector field can be related by a mathematical mapping wherein the mathematical mapping can be learnable by the deep neural network, wherein the network comprises a computer equipped with a GPU to train and deploy the neural network, wherein the neural network is constructed, trained and tested using a programming language and a machine learning library.

18. The method of claim 14 wherein the motion of a structure in the 3D volumetric image is determined based on the fixed 2D images that are continuously acquired during the procedure so that the procedure is focused on a patient's target organ while avoiding organs at risk.

19. The method of claim 17 wherein the deep neural network can be used to estimate both 3D DVFs and fixed 3D images at a moment of interest based on fixed 2D images acquired at the moment of interest, prior moving 2D images and a prior updated moving 3D image.

20. The method of claim 13 wherein the 3D volumetric image is an image selected from the group consisting of a computed-tomography image, a magnetic resonance image, a positron emission tomography image, a synthetic image, and an X-ray image, wherein the method can be carried out by accessing on-board imaging available on a standard linear accelerator.

21. The system of claim 1, wherein the deep neural network is further configured to accept auxiliary inputs comprising at least one of:
gantry angle information associated with acquisition of the 2D images;
optical surface imaging data of the patient's external anatomy; and
anatomical masks defining regions of interest.

22. A method for incorporating auxiliary inputs in image registration and volumetric imaging, the method comprising:
receiving at least one auxiliary input selected from:
gantry angle information;
optical surface imaging data; and
anatomical masks;
processing the auxiliary inputs through dedicated network pathways;
concatenating processed auxiliary inputs with internal network features; and
utilizing the concatenated features to improve accuracy of the image registration and volumetric imaging.
